(19) United States
(12) Patent Application Publication
(10) Pub. No.: US 2018/0000653 A1
(43) Pub. Date: Jan. 4, 2018

(54) APPARATUS AND A METHOD FOR FOLDING THE WINGS OF ABSORBENT SANITARY ARTICLES

(71) Applicant: Fameccanica.Data S.p.A., Pescara (IT)

(72) Inventors: Gabriele SABLONE, Pescara (IT); Antonio GALLUCCI, Spoltore (Pescara) (IT)

(21) Appl. No.: 15/631,850

(22) Filed: Jun. 23, 2017

(30) Foreign Application Priority Data
Jun. 30, 2016 (IT) 102016000068037

(51) Int. Cl. A61F 13/15; B65H 45/28; A61F 13/551; B65H 45/22; B65H 39/14

(52) U.S. Cl. CPC A61F 13/15747; A61F 13/5514; B65H 45/22; B65H 45/28; A61F 13/15577; B65H 39/14

(57) ABSTRACT

A folding apparatus for folding absorbent sanitary articles having an absorbent central body and two wings projecting from opposite side edges of said absorbent central body, comprising: a vacuum conveyor comprising a conveyor belt which holds the absorbent central bodies of said absorbent sanitary articles and moves said absorbent sanitary articles in a longitudinal direction, and a pair of stationary folding elements that engage respective wings of said absorbent sanitary articles while these are moved in said longitudinal direction by said vacuum conveyor, wherein said conveyor belt comprises a plurality of recessed seats which receive the absorbent central bodies of said absorbent sanitary articles, with said wings extending laterally outwards from said recessed seats on an outer surface of said conveyor belt.

### CROSS-REFERENCE TO RELATED APPLICATIONS

This application claims benefit of Italian patent application number 102016000068037, filed Jun. 30, 2016, which is herein incorporated by reference.

### BACKGROUND OF THE INVENTION

### Field of the Invention

The present invention relates to methods and apparatus for producing disposable absorbent sanitary articles.

More specifically, the invention relates to methods and apparatus for producing absorbent sanitary articles comprising an absorbent central body extending in a longitudinal direction and two lateral wings extending from opposite sides of the absorbent central body.

### Description of Prior Art

In the state of the art, female sanitary napkins are well-known, comprising an absorbent central body and a pair of wings extending from opposite sides of the absorbent central body. The wings are intended to be folded around the undergarment edges and to be stuck onto the outer side of the undergarment to retain the absorbent central body in the correct position during use.

For example, U.S. Pat. No. 4,285,343 describes an absorbent sanitary article with an absorbent central body and with two transversal wings that extend laterally from opposite parts of the absorbent central body. The wings can be formed as an integral part of the central body or can be formed separately and fixed to the longitudinal edges of the absorbent central body. The joining lines between the absorbent central body and the wings are flexible so that the wings can be folded onto the absorbent central body for packaging. During use, the wings are opened and folded around the edges of the undergarment and stuck to the outer side of the undergarment. The wings are usually provided with a layer of glue on their outer surface, for the adhesive attachment of the wings onto the outer side of the undergarment.

The most widely used methods for producing absorbent sanitary articles of this type are based on technology known in the sector as “machine direction”, which envisages forming a continuous chain of blanks of products oriented with their longitudinal axis aligned with the direction of movement of the continuous chain. Generally, absorbent sanitary articles of this type are formed with extended wings, coplanar to the absorbent central body. Before being packaged, the wings are folded, for example, on the inner surface of the absorbent central body, i.e. on the surface that is intended to come into contact with the body of the user, during use.

In the “machine direction” manufacturing technique, the folding of the wings on the surface, for example, on the inner surface of the absorbent central body, is carried out by means of a stationary folding device that, due to the movement of the absorbent sanitary articles in the machine direction, engages the wings and rotates them by 180° C. between the extended position and the folded position against the surface of the absorbent central body. WO2007/02919 describes a folding system for folding the wings that extend laterally from respective central bodies of a plurality of absorbent articles formed of a continuous composite web movable in a longitudinal direction. The stationary folding device comprises a pair of folding elements that engage the respective wings while the absorbent sanitary articles move in the longitudinal direction.

A folding device of the type described in WO2007/029195 requires that the folding lines of the two wings are spaced apart in a transverse direction by a distance equal to or greater than the maximum width of the absorbent central body. In the case where the transversal distance between the folding lines of the wings is less than the maximum width of the absorbent central body, the folding device intended to carry out the folding of the wings would also fold the parts of the absorbent central body located outside the folding lines. In cases where the absorbent central body has a generic hourglass shape with the wings located in the narrower part of the absorbent central body, the known types of folding devices are unable to fold the wings against the inner surface of the absorbent central body without folding the outer parts of the absorbent central body.

### SUMMARY OF THE INVENTION

The present invention aims to provide a method and an apparatus for folding wings of absorbent sanitary articles that allows the wings to be folded even when the distance between the folding lines is less than the maximum width of the absorbent central body.

According to the present invention, this object is achieved by a folding apparatus and method having the characteristics forming the subject of claims 1 and 7.

The claims form an integral part of the disclosure provided here in relation to the invention.

### BRIEF DESCRIPTION OF THE DRAWINGS

The present invention will now be described in detail with reference to the attached drawings, given purely by way of non-limiting example, wherein:

FIG. 1 is a schematic side view of a folding device according to the present invention.

FIG. 2 is a perspective view illustrating the part indicated by the arrow II in FIG. 1.

FIGS. 3 and 4 are plan views according to the arrow III of FIG. 1 showing the folding sequence of the wings.

FIGS. 5, 6, 7, 8 and 9 are cross-sections according to the lines V-V, VI-VI, VII-VII, VIII-VIII and IX-IX, respectively, of FIGS. 3 and 4.

FIG. 10 is a cross-section along the line X-X of FIG. 6.

### DETAILED DESCRIPTION

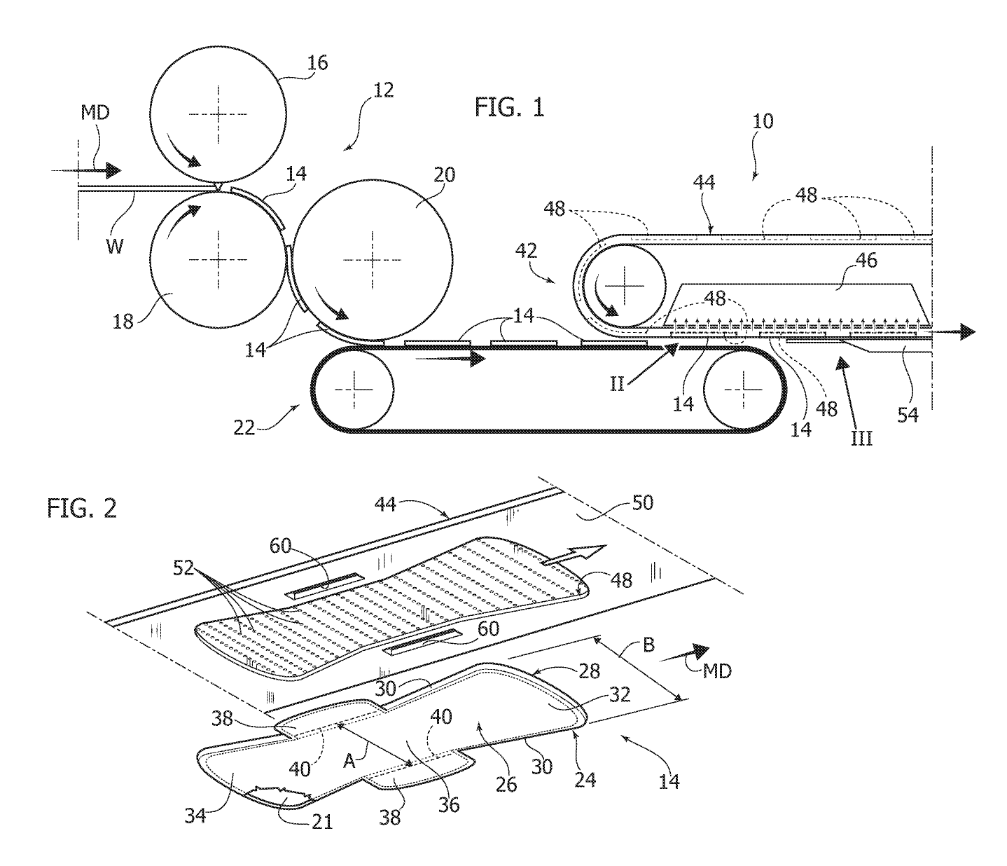

With reference to FIG. 1, 10 indicates a folding device for folding wings of disposable absorbent sanitary articles. The folding device 10 is arranged downstream of a cutting unit 12, which performs the cutting of a continuous composite web W in the machine direction MD. Following cutting, the continuous composite web W is subdivided into a continuous succession of absorbent sanitary articles 14, separated from each other. The cutting unit 12 comprises, in a conventional manner, a knives roller 16 and an anvil roller 18. After the cutting, the absorbent sanitary articles 14 can be retained by suction on the periphery of the anvil roller 18. Then, a detaching device 20, typically a suction roller, picks up the absorbent sanitary articles 14 from the cutting unit 12 and places them on a transfer belt 22, which transfers the absorbent sanitary articles to the folding device 10.

With reference to FIG. 2, each absorbent sanitary article 14 comprises an absorbent central body 24 having an upper surface 26 intended, during use, to come into contact with the body, and a lower surface 28 intended, during use, to come into contact with an undergarment. The absorbent central body 24 comprises an absorbent core 21 enclosed between a topsheet permeable to body fluids and a backsheet impermeable to body fluids. The absorbent central body 24 has two lateral sides 30 with an arcuate shape, which give the absorbent central body 24 a general hourglass shape, with two end portions 32, 34 wider than a central portion 36.

The absorbent sanitary article 14 comprises a pair of wings 38 located on opposite sides of the central absorbing body 24 and extending laterally outwards from the central portion 36 of the absorbent central body 24. The wings 38 are foldable relative to the central body 24 along respective folding lines 40. The shape of the absorbent central body 24 is such that the distance A between the folding lines 40—in an orthogonal direction to the direction of movement MD—is less than the maximum width B of the absorbent central body 24.

With reference to FIG. 1, the folding apparatus 10 comprises a vacuum conveyor 42 comprising a perforated conveyor belt 44 having a straight portion facing a suction box 46.

With reference to FIG. 2, the conveyor belt 44 has a plurality of recessed seats 48 configured for receiving respective absorbent central bodies 24 of absorbent sanitary articles 14. The recessed seats 48 have side walls with a shape corresponding to the side edges 30 of the absorbent central bodies 24, so that the wings 38 of the absorbent sanitary articles 14 extend outwardly from the recessed seats 48 and rest on the outer surface 50 of the conveyor belt 44. The recessed seats 48 are provided with holes 52 that cause the recessed seats 48 to communicate with the suction box 46 so that the absorbent central bodies 24 can be retained by suction in the respective recessed seats 48. The wings 38 can also be retained by suction in an extended position on the outer surface 50 of the conveyor belt 44. The depth of the recessed seats 48 is equal to or greater than the thickness of the absorbent central bodies 24, so that the upper surface 26 of the absorbent central bodies 24 is level with or in a lower position relative to the outer surface 50 of the conveyor belt 44.

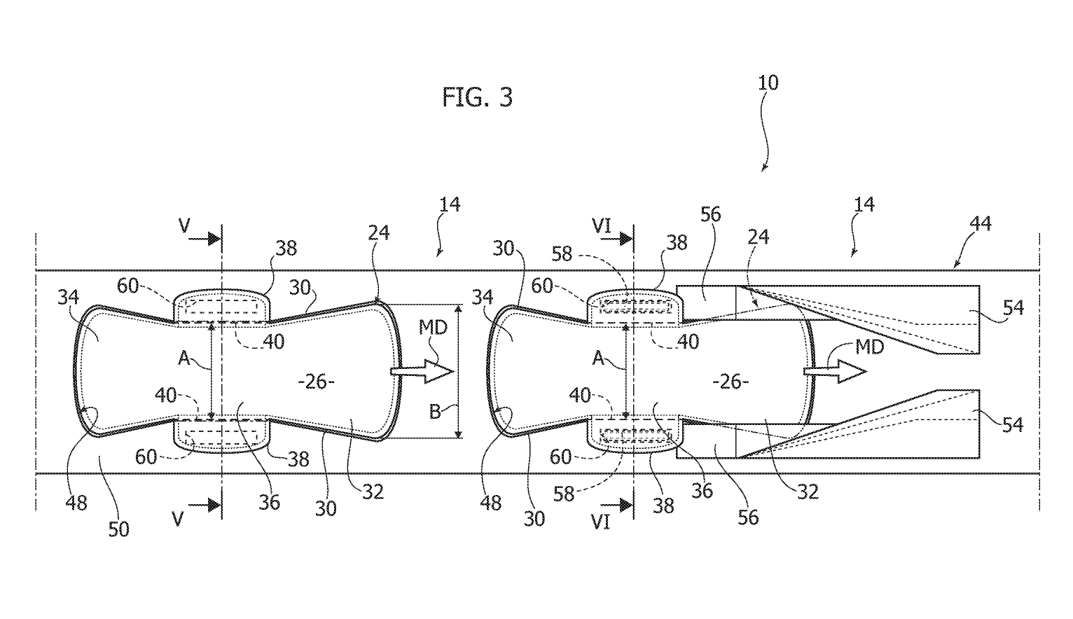

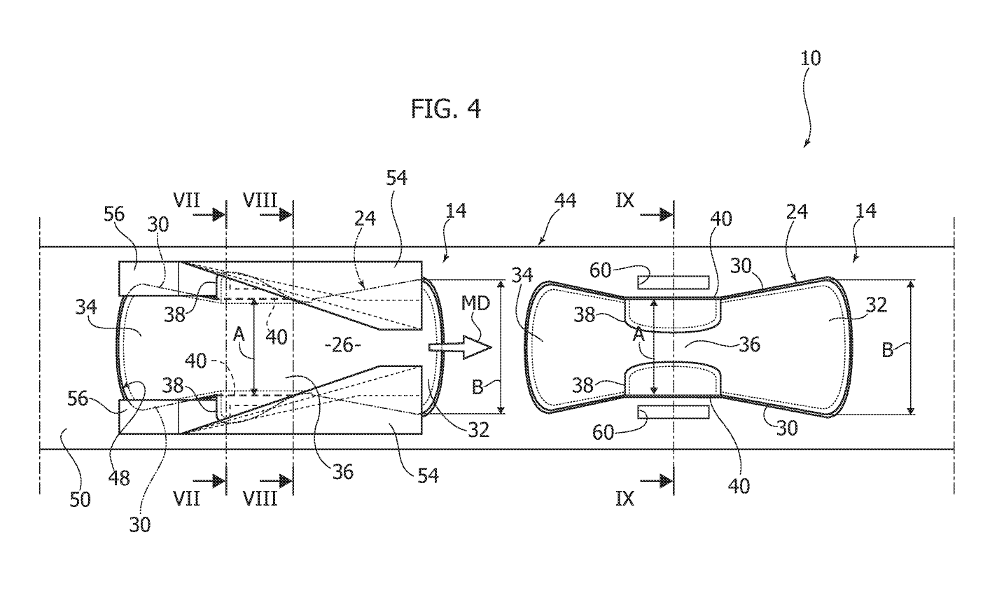

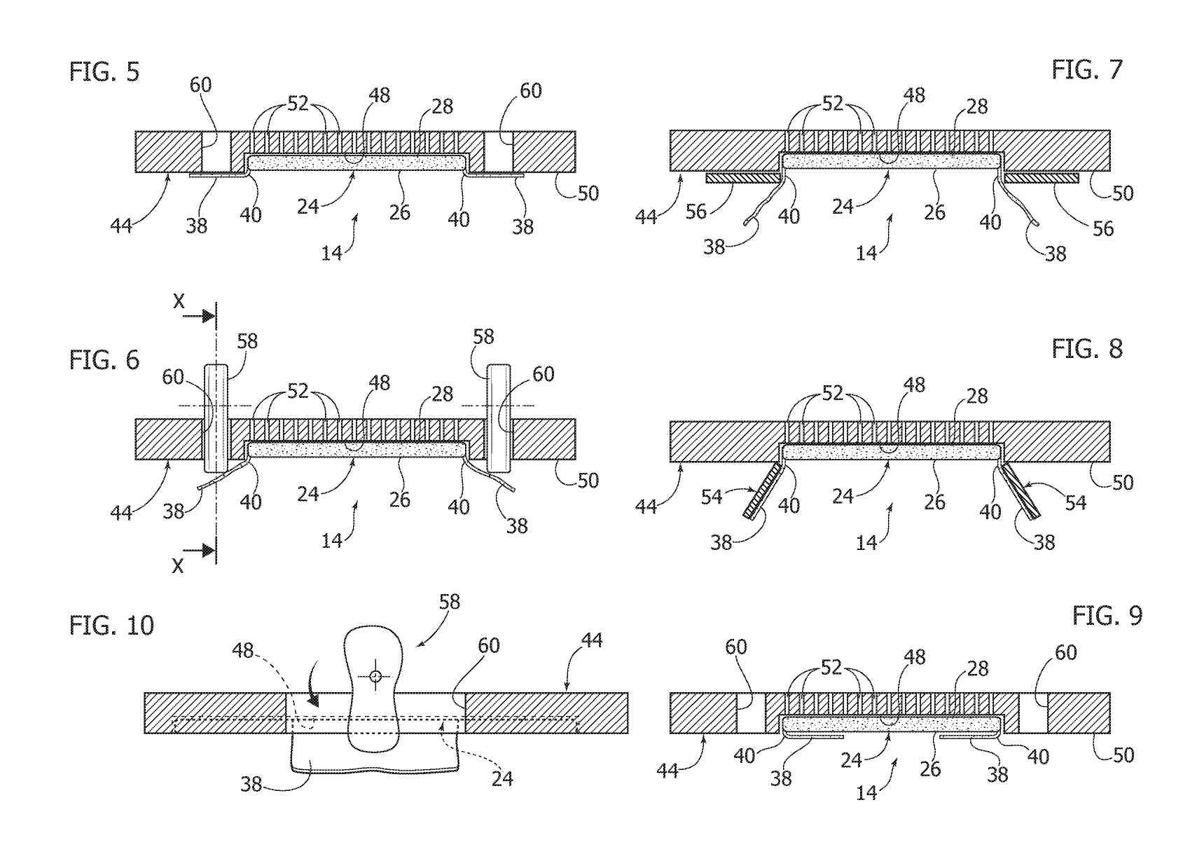

With reference to FIGS. 3 and 4, the folding apparatus comprises a pair of stationary folding elements 54 facing a straight portion of the conveyor belt 44. The folding elements 54 have respective ends 56 which are inserted between respective wings 38 and the outer surface 50 of the conveyor belt 44. The folding elements 54 have respective helical surfaces that, due to the movement of absorbent sanitary articles 14 in the longitudinal direction MD, fold the wings 38 of the absorbent sanitary articles 14 along the respective folding lines 40. The folding elements 54 are configured to carry out a rotation of 180° C. of the wings 38 around the respective folding lines 40, between a position in which the wings 38 extend outwardly to a position in which, in the illustrated example, the wings 38 are folded inside the respective absorbent central body 24 in contact with the respective upper surface 26.

With reference to FIGS. 6 and 10, the folding apparatus 10 can be provided with detaching means, which detach the wings 38 from the outer surface 50 of the conveyor belt 44 in the vicinity of the ends 56 of the folding elements 54. In the illustrated example, these detaching means comprise a pair of cams 58, rotatable about a transverse axis in phase with the conveyor belt 44, these cams can also be wheels with an eccentric rotational axis. The conveyor belt 44 is provided with through-slots 60 associated with each recessed seat 48, and located at the areas where the wings 38 are provided. The wheels 58 are inserted within respective through-slots 60 when the corresponding recessed seat 48 is located at the end 56 of the folding elements 54. The wheels 58 detach the wings 38 from the outer surface 50 of the conveyor belt 44 and facilitate the insertion of the ends 56 of the folding elements 54 between the outer surface 50 of the conveyor belt 54 and the wings 38, as shown in the right part of FIG. 3 and FIG. 7.

In the case in which the wings 38 protrude outwardly beyond the outer edges of the absorbent central body 24, the detaching means can be replaced by recessed grooves formed in the outer surface 50 of the conveyor belt 40, which allow the insertion of extension elements of the ends 56 of the folding elements 54 below the wings 38.

As can be seen in FIG. 7, the ends 56 of the folding elements 54 engage the wings 38 without interfering with the absorbent central bodies 24 of the absorbent sanitary articles 14. In fact, the wings 38, instead of being coplanar to their respective absorbent central bodies 24, by positioning the absorbent central bodies 24 in the respective lowered seats 48, they are spaced apart from the absorbent central bodies 24 in a direction orthogonal to the outer surface 50 of the conveyor belt 44. This arrangement allows the folding elements 54 to engage the wings 38 without interfering with the parts of the absorbent central bodies 24 located outside the folding lines 40.

Of course, without prejudice to the principle of the invention, the details of construction and the embodiments can be widely varied with respect to those described and illustrated, without thereby departing from the scope of the invention as defined by the claims that follow.

1. A folding apparatus for folding absorbent sanitary articles having an absorbent central body and two wings projecting from opposite side edges of said absorbent central body, comprising:
a vacuum conveyor comprising a conveyor belt, which holds the absorbent central bodies of said absorbent sanitary articles and moves said absorbent sanitary articles in a longitudinal direction; and
a pair of stationary folding elements that engage respective wings of said absorbent sanitary articles while they are moved in said longitudinal direction by said vacuum conveyor, wherein said conveyor belt comprises a plurality of recessed seats which receive the absorbent central bodies of said absorbent sanitary articles, said recessed seats having a shape such that said wings extend laterally outwards from said recessed seats on an outer surface of said conveyor belt.

2. An apparatus according to claim 1, wherein said recessed seats have a depth equal to or greater than the thickness of said absorbent central bodies.

3. An apparatus according to claim 1, wherein said recessed seats have side walls with a shape corresponding to the side edges of said absorbent central body.

4. An apparatus according to claim 1, wherein said recessed seats are provided with holes that cause said recessed seats to communicate with a suction box.

5. An apparatus according to claim 1, comprising a detaching device to detach said wings from said outer surface of the conveyor belt at the ends of said folding elements.

6. An apparatus according to claim 5, wherein said detaching device comprises a pair of cams rotating in phase with said conveyor belt and arranged to be inserted into respective through-slots formed in the conveyor belt at each recessed seat.

7. A method for folding absorbent sanitary articles having an absorbent central body and two lateral wings projecting from opposite side edges of said absorbent central body, comprising the steps of:
moving said absorbent sanitary articles in a longitudinal direction using a vacuum conveyor comprising a conveyor belt; and
engaging the wings of said absorbent sanitary articles by respective stationary folding elements while the sanitary absorbent articles are moved in said longitudinal direction by said folding conveyor, wherein said absorbent central bodies of said sanitary absorbent articles are received within recessed seats of said conveyor belt and said lateral wings extend laterally outwards from said recessed seats on an outer surface of said conveyor belt.

8. A method according to claim 7, wherein said recessed seats have a depth equal to or greater than the thickness of said absorbent central bodies.

9. A method according to claim 7, comprising the step of detaching said wings from said outer surface of said conveyor belt prior to engaging said wings with said folding elements.
